(19) United States
(12) Patent Application Publication
(10) Pub. No.: US 2025/0064572 A1
(43) Pub. Date: Feb. 27, 2025

(54) Vascular Implant

(71) Applicant: BARD PERIPHERAL VASCULAR, INC., Franklin Lakes, NJ (US)

(72) Inventors: Matt Casiraro, Tempe, AZ (US); Alexander Lastovich, Gilbert, AZ (US)

(73) Assignee: BARD PERIPHERAL VASCULAR, INC., Franklin Lakes, NJ (US)

(21) Appl. No.: 18/724,268

(22) PCT Filed: Dec. 30, 2021
(86) PCT No.: PCT/US2021/073181

§ 371 Date: Jun. 26, 2024

(51) Int. Cl. A61F 2/01

(52) U.S. Cl. CPC A61F 2/01; A61F 2210/0004; A61F 2220/0025; A61F 2230/0019

(57) ABSTRACT

A vascular implant body having opposed filter ends and a central longitudinal axis. The implant body has a segmented side wall comprised of spaced apart wall panels that surround an open-ended bore that extends along the central longitudinal axis. Multiple filtering elements extend from a side wall to a core of the filter body, some of the filtering elements terminating at the core where they connect to another filtering element. The filtering elements can form an acute angle with the central longitudinal axis. One or more filtering elements extend from a first position on the side wall to a second position on the side wall that is spaced from the first position.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

### 1 Field of the Invention

The present invention relates to bioresorbable vascular implants such as filters (e.g., vena cava filters), occlusion devices, stents and convertible stents. In an embodiment, the present invention relates to an improved vascular implant that bioresorbs into a patient's vascular system (e.g., inferior vena cava filter, iliofemoral vein, ovarian vein, splenic artery, uterine artery, hepatic artery or other vein/artery vessel). For the inferior vena cava and iliofemoral vein, the implant may resorb after transient risk of pulmonary embolism (PE) has subsided. In preferred embodiments of the present invention, the entire implant structure could be made of bioresorbable material so that no implant or implant remnant/element would ultimately be left behind as the entire implant would preferably resorb into the vascular tissue. In one embodiment of the present invention, the implant body can have a tubular shape and is preferably comprised of a plurality of circumferentially spaced apart wall panels connected together with filtering elements that each form an acute angle with a filter body central, longitudinal axis.

### 2. General Background of the Invention

Vascular implants include various devices that are placed at a selected locale in a patient's blood vessel. One example is a vena cava filter. Other examples include occlusion devices, stents and convertible stents. Various patents have issued for vascular implants. Patents have also issued that relate in general to 3d printing of implants. Examples are listed in the following Table 1. Each patent or publication listed in Table 1 is hereby incorporated herein by reference.

TABLE 1

| PATENT OR | | |
|---|---|---|
| PUBLICATION | | ISSUE DATE |
| NO. | DESCRIPTION | (DD/MM/YYYY) |
| 8,092,484 | EMBOLUS BLOOD CLOT FILTER WITH | 10 Jan. 2012 |
| | POST DELIVERY ACTUATION | |
| 8,092,485 | RECOVERABLE INFERIOR VENA CAVA | 10 Jan. 2012 |
| | FILTER | |
| 8,317,818 | REMOVABLE BLOOD CLOT FILTER WITH | 27 Nov. 2012 |
| | EDGE FOR CUTTING THROUGH THE | |
| | ENDOTHELIUM | |
| 8,420,113 | BIODEGRADABLE MEDICAL DEVICES | 16 Apr. 2013 |
| | WITH ENHANCED MECHANICAL | |
| | STRENGTH AND PHARMACOLOGICAL | |
| | FUNCTIONS | |
| 8,518,072 | JUGULAR FEMORAL VENA CAVA FILTER | 27 Aug. 2013 |
| | SYSTEM | |
| 8,562,638 | EMBOLUS BLOOD CLOT FILTER WITH | 22 Oct. 2013 |
| | FLOATING FILTER BASKET | |
| 8,734,479 | EMBOLUS BLOOD CLOT FILTER | 27 May 2014 |
| | DELIVERY SYSTEM | |
| 8,777,975 | EMBOLUS BLOOD CLOT FILTER WITH | 15 Jul. 2014 |
| | BIO-RESORBABLE COATED FILTER | |
| | MEMBERS | |
| 8,795,351 | MIGRATION RESISTANT EMBOLIC | 5 Aug. 2014 |
| | FILTER | |
| 8,870,943 | STENT STRUCTURE FOR IMPLANTATBLE | 28 Oct. 2014 |
| | MEDICAL DEVICE | |
| 8,992,562 | FILTER DELIVERY SYSTEM | 31 Mar. 2015 |
| 9,220,588 | SYSTEMS, METHODS AND DEVICE FOR | 29 Dec. 2015 |
| | EMBOLIC PROTECTION | |
| 9,393,095 | JUGULAR FEMORAL VENA CAVA FILTER | 19 Jul. 2016 |
| | SYSTEM | |
| 9,421,081 | EMBOLUS BLOOD CLOT FILTER | 23 Aug. 2016 |
| | DELIVERY SYSTEM | |
| 9,445,895 | INTRACARDIAC CAGE AND METHOD OF | 25 Sep. 2016 |
| | DELIVERING SAME | |
| 9,456,888 | REVERSIBLE VASCULAR FILTER | 4 Oct. 2016 |
| | DEVICES AND METHODS FOR USING | |
| | SAME | |
| 9,468,513 | EMBOLUS BLOOD CLOT FILTER WITH | 18 Oct. 2016 |
| | BIO-RESORBABLE COATED FILTER | |
| | MEMBERS | |
| 9,561,094 | DEVICES AND METHODS FOR TREATING | 7 Feb. 2017 |
| | VENOUS DISEASES | |
| 9,597,435 | MEDICAL DEVICES HAVING A | 21 Mar. 2017 |
| | BIORESORBABLE COATING LAYER WITH | |
| | A PRE-DETERMINED PATTERN FOR | |
| | FRAGMENTATION | |
| 9,693,851 | FILTER DELIVERY SYSTEM | 14 Jul. 2017 |
| 9,730,781 | EMBOLUS BLOOD CLOT FILTER | 15 Aug. 2017 |
| | REMOVAL SYSTEM AND METHOD | |
| 9,949,816 | IVC FILTER RETRIEVAL SYSTEMS WITH | 24 Apr. 2018 |
| | MULTIPLE CAPTURE MODES | |
| 9,980,804 | VENA CAVA FILTER WITH FILAMENT | 29 May 2018 |
| 10,105,206 | INFERIOR VENA CAVA FILTER WITH | 23 Oct. 2018 |
| | STABILITY FEATURES | |
| 10,188,496 | VENA CAVA FILTER FORMED FROM A | 29 Jan. 2019 |
| | SHEET | |
| 10,188,498 | EMBOLUS BLOOD CLOT FILTER | 29 Jan. 2019 |
| | DELIVERY SYSTEM | |
| 10,226,322 | JUGULAR FEMORAL VENA CAVA FILTER | 12 Mar. 2019 |
| | SYSTEM | |
| 10,258,454 | VISUAL STABILIZER ON ANCHOR LEGS | 16 Apr. 2019 |
| | OF VENA CAVA FILTER | |
| 10,279,078 | CROSSLINKABLE 3D PRINTED | 7 May 2019 |
| | BIOMATERIAL-BASED IMPLANTS AND | |
| | METHODS OF MANUFACTURE THEREOF | |
| 10,299,906 | EMBOLUS BLOOD CLOT FILTER | 28 May 2019 |
| | UTILIZABLE WITH SINGLE DELIVERY | |
| | SYSTEM OR A SINGLE RETRIEVAL | |
| | SYSTEM IN ONE OF A FEMORAL OR | |
| | JUGULAR ACCESS | |
| 10,342,654 | IVC FILTER WITH TRANSLATING HOOKS | 9 Jul. 2019 |
| 10,368,972 | EMBOLUS BLOOD CLOT FILTER WITH | 6 Aug. 2019 |
| | BIO-RESORBABLE COATED FILTER | |
| | MEMBERS | |
| 10,390,925 | MIGRATION RESISTANT EMBOLIC | 27 Aug. 2019 |
| | FILTER | |
| 10,441,689 | METHODS AND DEVICES FOR THREE- | 15 Oct. 2019 |
| | DIMENSIONAL PRINTING OR ADDITIVE | |
| | MANUFACTURING OF BIOACTIVE | |
| | MEDICAL DEVICES | |
| 10,470,865 | VASCULAR FILTER DEVICE | 12 Nov. 2019 |
| 10,492,898 | EMBOLUS BLOOD CLOT FILTER AND | 3 Dec. 2019 |
| | DELIVERY SYSTEM | |
| 10,512,531 | FILTER DELIVERY SYSTEM | 24 Dec. 2019 |
| 10,531,942 | ABSORBABLE VASCULAR FILTER | 14 Jan. 2020 |
| 10,579,755 | METHOD FOR 3-D PRINTING A CUSTOM | 3 Mar. 2020 |
| | BONE GRAFT | |
| 10,624,731 | VASCULAR FILTER SYSTEM | 21 Apr. 2020 |
| 10,729,527 | REMOVABLE EMBOLUS BLOOD CLOT | 4 Aug. 2020 |
| | FILTER | |
| 10,813,738 | TUBULAR FILTER | 27 Oct. 2020 |
| 10,842,608 | VENA CAVA FILTER WITH FILAMENT | 24 Nov. 2020 |
| 2007/0064731 | TRANSMISSION APPARATUS WITH | 22 Mar. 2007 |
| | FUNCTION OF MULTI-STEP BANDWIDTH | |
| | ASSIGNMENT TO OTHER | |
| | COMMUNICATION APPARATUSES | |
| 2010/0074934 | MEDICAL IMPLANTS WITH A | 25 Mar. 2010 |
| | COMBINATION OF COMPOUNDS | |
| 2016/0166371 | ENDOLUMINAL FILTER DESIGN | 16 Jun. 2016 |
| | VARIATIONS | |
| 2016/0175085 | ENHANCED FLUOROGENIC | 23 Jun. 2016 |
| | ENDOLUMINAL FILTER STRUCTURE | |
| 2017/0105830 | BIODEGRADABLE VASCULAR FILTER | 20 Apr. 2017 |
| 2017/0218228 | THREE DIMENSIONAL PRINTING OF BIO- | 3 Aug. 2017 |
| | INK COMPOSITIONS | |
| 2017/0249440 | 3D PRINTING SURGICAL REPAIR | 31 Aug. 2017 |
| | SYSTEMS | |
| 2017/0340429 | VASCULAR FILTER SYSTEM | 30 Nov. 2017 |
| 2018/0168811 | NOVEL BIODEGRADABLE AND NON- | 21 Jun. 2018 |
| | BIODEGRADABLE 3D PRINTED | |
| | IMPLANTS AS A DRUG DELIVERY | |
| | SYSTEM | |
| 2018/0296343 | 3-D PRINTING OF POROUS IMPLANTS | 18 Oct. 2018 |
| 2018/0303616 | 3-D PRINTING OF BONE GRAFTS | 25 Oct. 2018 |
| 2018/0311028 | VENA CAVA FILTER WITH FILAMENT | 1 Nov. 2018 |
| 2019/0110880 | MEDICAL DEVICES AND ANCHORS | 18 Apr. 2020 |
| | THREFOR | |
| 2020/0001540 | ADDITIVE MANUFACTURING ON | 2 Jan. 2020 |
| | UNCONSTRAINED FREEFORM SURFACES | |
| 2020/0197150 | VASCULAR FILTER SYSTEM | 25 Jun. 2020 |
| WO2007064731 | HELICAL VENA CAVA FILTER | 7 Jun. 2007 |
| WO2011079287 | REVERSIBLE VASCULAR FILTER | 30 Jun. 2011 |
| | DEVICES AND METHODS FOR USING | |
| | SAME | |
| WO2016154148 | ARTIFICIAL TYMPANIC MEMBRANE | 29 Sep. 2016 |
| | DEVICES AND USES | |
| WO2018117907 | SHAPE MEMORY POLYMER COMPOSITE | 28 Jun. 2018 |
| | FOR 3D PRINTING OF MEDICAL ITEMS | |
| WO2018218085 | THREE-DIMENSIONAL PRINTED | 29 Nov. 2018 |
| | ORGANS, DEVICES, AND MATRICES | |
| WO2019178086 | ELECTROHYDRODYNAMIC BIOPRINTER | 19 Sep. 2019 |
| | SYSTEM AND METHOD | |
| WO2020123945 | FABRIC MATERIAL FOR MEDICAL | 18 Jun. 2020 |
| | DEVICES | |
| EP2363156 | METHOD OF FABRICATING | 7 Sep. 2011 |
| | BIODEGRADABLE MEDICAL DEVICES | |
| | WITH ENHANCED MECHANICAL | |
| | STRENGTH AND PHARMACOLOGICAL | |
| | FUNCTIONS | |

### BRIEF SUMMARY OF THE INVENTION

The present invention provides a vascular implant preferably having opposed filter ends and a central longitudinal axis.

In one or more preferred embodiments, the body preferably has a generally tubular side wall surrounding a central, open ended bore.

In one or more embodiments, multiple filtering elements preferably extend from one position on the side wall to another, spaced apart position on the side wall.

In one or more preferred embodiments, the filtering elements preferably form an acute angle with the central longitudinal axis.

In one or more embodiments, the filtering elements preferably extend from a first position on the side wall to a second position on the side wall that is preferably spaced from the first position.

In one or more preferred embodiments, the implant body is preferably a 3d printed body.

In one or more embodiments, one or more of the filtering elements preferably do not touch one or more others of the filtering elements.

In one or more preferred embodiments, one or more of the filtering elements preferably attaches to the side wall at a position that is close to the upper end and to the side wall at a position that is closer to the lower end.

In one or more preferred embodiments, multiple filtering elements are preferably attached to the side wall at positions on the side wall less than 180 degrees apart.

In one or more preferred embodiments, the side wall is preferably a segmented side wall comprised of multiple circumferentially spaced apart wall sections.

In one or more preferred embodiments, at least one wall section preferably has a generally rectangular shape.

In one or more preferred embodiments, each wall section preferably has a generally rectangular shape.

In one or more preferred embodiments, the opposed filter ends preferably include a proximal end and a distal end.

In one or more preferred embodiments, the body is preferably of a material that is too soft to be machined.

In one or more preferred embodiments, the implant body preferably has a durometer reading of between 35 Shore A and 75 Shore D.

In one or more preferred embodiments, the implant body is preferably of a resorbable material that resorbs when exposed to the human body vascular system. In one or more preferred embodiments, the implant body is preferably of bioresorbable polymer material. When of a bioresorbable material, the bioresorption starts as soon as the implant is exposed to the blood and like many of the standard polymers, degrades over time via polymer breakdown and absorption of the byproducts. This bioresorption can be tuned as needed to ensure the implant maintains structural filtering integrity until transient risk of pulmonary embolism has subsided.

In one or more preferred embodiments, one or more filtering elements preferably connect to the side wall and another filtering element.

In one or more preferred embodiments, the implant body preferably has opposed filter ends and a central longitudinal axis.

In one or more preferred embodiments, the implant body preferably has a segmented side wall comprised of spaced apart wall panels that surround an open-ended bore that preferably extends along the central longitudinal axis.

In one or more embodiments, multiple filtering elements preferably extend from the side wall to a core of the implant body, some of the filtering elements terminating at the core where they connect to another filtering element.

In one or more preferred embodiments, one or more filtering elements preferably extend from a first position on the side wall to a second position on the side wall that is preferably spaced from the first position.

In one or more preferred embodiments, the implant body is preferably of a resorbable material which resorbs when exposed to the human body vascular system.

In one or more preferred embodiments, a tubular implant body preferably has opposed filter ends and a central longitudinal axis.

In one or more preferred embodiments, there is preferably a tubular implant body having opposed filter ends and a central longitudinal axis.

In one or more preferred embodiments, the implant body preferably has a segmented side wall comprised of spaced apart wall panels that preferably surround an open-ended bore that extends along the central longitudinal axis.

In one or more preferred embodiments, the implant body preferably has a core portion spaced between the opposed filter ends and spaced inwardly of the segmented side wall.

In one or more embodiments, multiple filtering elements preferably extend from the side wall to the core of the implant body, some of the filtering elements terminating at the core where they connect to another filtering element.

In one or more preferred embodiments, one or more filtering elements preferably extend from one wall segment to another of the wall segments.

In one or more preferred embodiments, the filtering members may be interwoven, for example a first plurality of filtering members could overlap a second plurality.

In one or more preferred embodiments, the filtering members may form a woven mesh-like structure, such as wherein a first plurality of filtering members are woven with a second plurality of filtering members.

In one or more preferred embodiments, the heights of the filtering members may be staggered to prevent collision of members forming mesh-like structure.

In one or more preferred embodiments, when the vascular implant is at the lower end of the durometer range, for example from 10 Shore A to 40 Shore A, the filtering members may flex slightly as they pass above and below each other. While FIGS. 1-12 depict filtering members which are straight, in various embodiments the filtering members are able to bend slightly as they cross each other, forming a woven-like filtering structure, for example, as seen in FIGS. 15-17.

In one or more preferred embodiments, each filtering member may have a rounded, enlarged end or ball at each end to prevent each wall panel from separating from the implant.

In one or more preferred embodiments, one or more wall panels may comprise a socket which allows each filtering member end to pivot and move axially, but which prevents the filtering member from moving in a radial direction.

In one or more embodiments, each filtering member can pass through a hole or socket which may preferably be conically or frusto-conically shaped.

In one or more embodiments, when the vascular implant does not have any ball and socket connections, the vascular implant is preferably on the lower end of the durometer range, for example, when the material has a hardness that is within a durometer reading of about 10 Shore A and 40 Shore A.

In one or more preferred embodiments, when the vascular implant possesses ball and socket features, the vascular implant can be of a material that has a hardness within a durometer reading of about 10 Shore A and 75 Shore D.

In one or more preferred embodiments, the vascular implant can be of a material that has a hardness within a durometer reading of between about 65 Shore A and 75 Shore D

In one or more preferred embodiments, each wall panel can be connected laterally to the adjacent wall panels.

In one or more preferred embodiments, each wall panel can be connected to adjacent wall panels via interlocking portions.

In one or more preferred embodiments, each wall panel possesses at least one protrusion and at least one recess for interlocking with adjacent panels.

In one or more preferred embodiments, each wall panel can be of a similar dimension.

In one or more preferred embodiments, various wall panels can be of dissimilar dimensions.

In one or more preferred embodiments, each wall panel can be connected to one or more opposing wall panels via filter members or struts.

In one or more preferred embodiments, each filtering member connection with opposing wall panels can feature a ball and socket connection.

In one or more preferred embodiments, each filtering member can pass through a conically or frusto-conically shaped hole in each wall panel which it connects.

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is a side elevation view of a first embodiment of the apparatus of the present invention;

FIG. 2 is a top view of a first embodiment of the apparatus of the present invention;

FIG. 3 is a partially exploded, perspective view of a second embodiment of the apparatus of the present invention;

FIG. 4 is a perspective view of a second embodiment of the apparatus of the present invention;

FIG. 5 is a perspective view of a second embodiment of the apparatus of the present invention;

FIG. 6 is a view of two interlocking wall panels or wall segments connected together of a second embodiment of the apparatus of the present invention;

FIG. 7 is a view of two interlocking wall panels or wall segments connected together which are different than the interlocking wall panels or wall segments of FIG. 6 of a third embodiment of the apparatus of the present invention;

FIG. 8 is a close up, partial view of a wall panel and filtering member/strut of a second embodiment of the apparatus of the present invention;

FIG. 9 is a perspective view of two panels of a third embodiment of the apparatus of the present invention;

FIG. 10 is a partial, side cutaway view of a wall panel socket, filtering member and filtering member end of preferred embodiments of the present invention;

FIG. 11 is a perspective view of a fourth embodiment of the apparatus of the present invention;

FIG. 12 is a top view of the fourth embodiment shown in FIG. 11;

FIG. 13 is a perspective view of the fourth embodiment of the apparatus of the present invention;

FIG. 14 is a perspective view of the fourth embodiment of the apparatus of the present invention;

FIG. 15 is a top, partially exploded view of a fifth embodiment of the apparatus of the present invention;

FIG. 16 is a top, partially exploded view of a sixth embodiment of the apparatus of the present invention; and

FIG. 17 is another top view of the sixth embodiment of the apparatus of the present invention shown in FIG. 15.

### DETAILED DESCRIPTION OF THE INVENTION

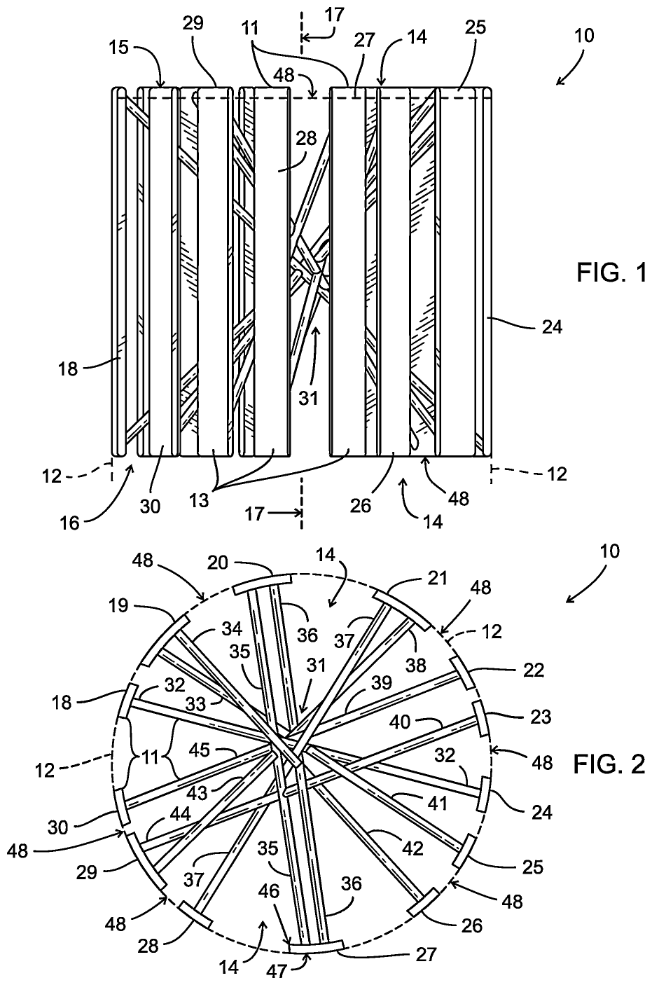

FIGS. 1 and 2 show a first embodiment of the apparatus of the present invention designated generally by the numeral 10. Vascular implant 10 has implant body 11 that can be of a polymeric or resorbable material such as a bioresorbable polymeric material.

In FIGS. 1 and 2, vascular implant 10 is shown in its deployed, expanded position. Implant body 11 includes a side wall, in this embodiment a segmented wall, side wall or tubular wall 13. Implant body 11 can be placed in a vessel, in which case the outer surface of the wall 13 contacts the vascular tissue 12, as illustrated with the dashed lines in FIGS. 1 and 2. Body 11 has an open ended bore or passageway 14. The side wall extends in circumferential direction around the central longitudinal axis 17. The side wall extends in axial direction between opposed filter ends, in this embodiment from the proximal end portion 15 to the distal end portion 16 and defines and encloses, in circumferential direction around the central longitudinal axis 17, the open-ended bore or passageway 14. Through the open-ended bore or passageway 14 fluid, e.g. blood, transported through the vessel, can flow, e.g. in the direction from the proximal end portion 15 to the distal end portion 16 (or vice versa). The passage may also be described as a central, open-ended bore, which is surrounded by the side wall 13.

Body 11 has first end portion 15 and second end portion 16. End portion 15 can be a proximal end portion. End portion 16 can be a distal end portion. Body 11 has a central longitudinal axis 17. Body 11 can be a vena cava filter, occlusion device, or convertible stent, for example. Body 11 can have a diameter of between about 15 and 30 mm for inferior vena cava pulmonary embolism prevention, a diameter of about 6-20 mm for iliofemoral pulmonary embolism prevention and a diameter of between 2-8 mm for other smaller vessels for occlusion. Body 11 can be 5 to 50 mm long.

In this embodiment, side wall 13 is generally tubular. The segmented side wall composed of multiple wall panels 18-30. Each wall panel or segment 18-30 can be generally rectangular in shape or any other desired shape. Each of wall panel or segment 18-30 can be planar and be flat or curved, such that the wall panel or segment 18-30 together form a segmented, polygonal cylindrical side wall or a circle cylinder side wall, for instance. Panels or sections 18-30 are circumferentially spaced as seen in FIGS. 1-2 with a gap 48 in between each pair of panels or wall sections 18-30. In this embodiment, the wall panels or wall segments 18-30 are oriented parallel to the central longitudinal axis. The segments are in this embodiment oriented in “portrait” mode, with their lateral sides extending in the longitudinal direction of implant body 11. Alternatively or additionally, some or all of the segments may be oriented in “landscape” mode, with their transverse sides extending in the longitudinal direction of implant body 11. The wall panels or wall segments 18-30 have an inwards facing surface which faces towards the central, longitudinal axis 17, and an outward facing surface which faces away from the central axis and forms the outer surface of the side wall 13. As shown, the wall panels or segments 18-30 may be oriented with their outwards facing surface extending parallel to the tangential direction, and the wall panels or segments 18-30 are in this embodiment located in radial direction at the same distance from the central axis 12, thus forming a side wall shaped as a segmented cylinder, which in this example is an open cylinder. The cylinder extends from the bottom, formed by a short side of the segments to the top, formed by the opposite short side of the segments.

As explained below in more detail, the body 11 comprises one or more filtering members 32-45 which block particles, e.g. blood clots, above a predetermined size from passing from one end through the passageway 14 to the opposite end, while allowing fluid to pass through the passageway 14. In this embodiment, the filtering elements 32-45 are located in the passageway 14, that is inside the central, open-ended bore. The filtering elements block particles that entered the passageway 14 at a respective open end from leaving the passageway at the, in the direction of fluid flow, opposite open end. In a preferred embodiment, the filtering elements attach the wall sections or panels to each other. That is, the filtering elements 32-45 permanently hold the wall sections or panels 18-30 in position relative each other and provide structural integrity to the implant body 11, such that a single mechanical body is obtained. The struts 32-45 can overlap one another as seen in FIGS. 1-2. Some struts can be connected to other struts. By connecting some struts to other struts and by overlapping the struts 32-45, the panels 18-30 are maintained in the positions shown in FIGS. 1-2. However, the struts 32-45 can bend if the panels move inwardly into a smaller diameter than the diameter of FIG. 2 such as when the implant 10 body 11 is collapsed to fit within a catheter. In such a collapsed position, the gap 48 would be smaller than gap 48 shown in FIGS. 1-2. In FIGS. 1-2, some of the struts 32-45 are closer to end 15 while others of the struts are closer to end 16.

In the embodiment of FIGS. 1-2, the filtering elements are filtering members or struts 32-45. As can be seen in FIG. 2, the filtering members or struts 32-45 extend from a position on the side wall 13 through the central, open ended bore to a termination and partition the cross-sectional area of the passageway in a number of smaller, open areas or voids. Each open area or void is bound by one or more of the filtering members or struts 32-45 and/or the sidewall 13. When the implant is placed in a vessel, with the fluid flow being from a respective end portion through the passageway 14 to the other end portion, the fluid will stream through the open areas or voids between the filtering members or struts 32-45, and blood clots above a size determined by the dimensions of the open areas or voids between the filtering members or struts 32-45 cannot pass through the implant body.

A plurality of filtering members or struts 32-45 each extend from a panel 18-30 toward a central region or core 31, as seen in FIGS. 1-2. Filtering members or struts 32-45 can be of a bioresorbable polymer material. Strut length can be between about 5 and 50 mm. Some of these filtering members or struts 32-45 can be longer and extend from one panel or wall section 18-30 to another panel or wall section 18-30. For example, in FIG. 2: filtering member or strut 32 extends from wall section or panel 18 to wall section or panel 24; filtering member or strut 35 extends from wall section or panel 20 to wall section or panel 27; filtering member or strut 36 extends from wall section or panel 20 to wall section or panel 27; and filtering member or strut 37 extends from wall section or panel 21 to wall section or panel 28. Some or all of the filtering members or struts may pass through, or terminate, at the axis 17. Alternatively, or additionally, some or all of the filtering members or struts may be a chord not passing through or terminating at the axis 17.

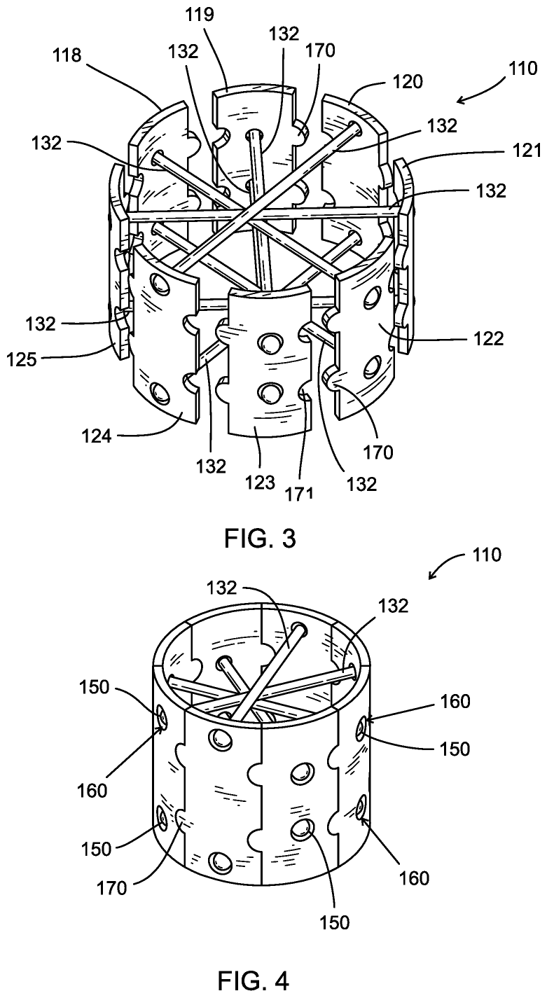

As can be seen in FIG. 2, some or all of the filtering members or struts may, seen in a cross-sectional view, cross-each other, forming a woven-like filtering structure. Some of the filtering members or struts may be skewed. Some of the filtering members or struts may be extend in the same plane or in parallel planes. Some of the filtering members or struts extending in the same plane may be parallel to each other (Filtering member or strut 35 is parallel to filtering member or strut 36, for example). Some of the filtering members or struts extending in the same, or parallel, plane may be non-parallel to each other (In FIG. 3, filtering members or struts 132 extend in parallel planes but are not parallel to each other).

Each filtering member or strut 32-45 preferably forms an acute angle with axis 17. Each filtering member or strut 32-45 preferably extends in a direction from first end portion 15 towards second end portion 16, e.g. in the longitudinal direction of the passageway but not parallel thereto. In one preferred embodiment, each filtering member or strut 32-45 connects to a wall section or panel 18-30 next to either first end portion 15 or second end portion 16. The filtering members or struts 32, 35, 36, 37 each preferably connect to a wall section or panel next to first end portion 15 and also to a wall section or panel at second end portion 16. In one preferred embodiment, each filtering member or strut 32, 35, 36, 37 has one end connecting to a wall section or panel and another end connected to another wall section or panel. Preferably, the wall section or panel and the other wall section or panel are circumferentially separated by one or more other wall sections or panels. In a further preferred embodiment, each of the remaining filtering members or struts 33, 34, 38-45 connects to a wall section or panel 18-30 and to another filtering member 32-45 as seen in FIGS. 1-2. As an example, filtering member 34 connects to filtering member 36 at core or central region 31. Similarly, filtering member 41 connects to filtering member 32 at core or central region 31. Filtering member 43 connects to filtering member 35 at core or central region 31.

In the embodiment shown in FIGS. 1 and 2, each wall section or panel 18-30 is connected to one or more filtering member or struts 32-45. Some or all wall sections or panels 18-30 may be connected to one filtering member or strut 32-45. Alternatively, or additionally, some wall sections or panels 18-30 can be connected to two filtering members such as panel 19 (panel 19 connects to filtering members or struts 33-34), panel or wall section 20 (panel 20 connects to filtering members or struts 35-36), wall section 21 (wall section 21 connects to filtering members or struts 37, 38) and wall section or panel 27 (panel 27 connects to filtering members or struts 35, 36).

Each wall section or panel 18-30 can have a concave inner surface 46 and a convex outer surface 47 (see FIG. 2). The panels 18-30 can reduce the risk of damage to the vessel and/or reduce perturbations in the flow profile. Surfaces 46, 47 may be curved in the circumferential direction and not in the axial direction of the implant body 11. Preferably, but not necessarily, the curvature of the outer convex surface 47 and/or inner concave surface 46 in the circumferential direction is more or less the same as of a cylinder with a radius equal to the distance of the respective surface to the central longitudinal axis 17.

The implant 10 of FIGS. 1 and 2 is preferably of a durometer that is low enough to allow compression into a catheter to traverse to position but high enough that the device has enough rigidity to return to its uncompressed state in-vivo. For example, the implant 10 of FIGS. 1 and 2 can have a hardness that is within a durometer reading of about 10 Shore A and 40 Shore A. Preferably, the struts of implant 10 have a memory which allows them to return to the expanded position seen in FIGS. 1 and 2, after deployment.

The implant 10 could be deployed with either a femoral or jugular approach. Such a deployment could employ a pusher or pusher apparatus/mechanism such as one specified in one or more of the patents listed in Table 1. An example is U.S. Pat. No. 8,518,072 naming Jonathan Miller as inventor and assigned to C.R. Bard, Inc. Implant 10 could also be a balloon-mounted implant that is then expanded with balloon dilation, as seen, for example, in balloon expandable stents.

FIGS. 3-6 and 8 show a second, preferred embodiment of the apparatus of the present invention designated generally by the numeral 110. Except as noted and/or as depicted in the Figures, the embodiment of FIGS. 3-6 and 8 shares the features of the embodiment depicted in FIGS. 1 and 2 and previously described.

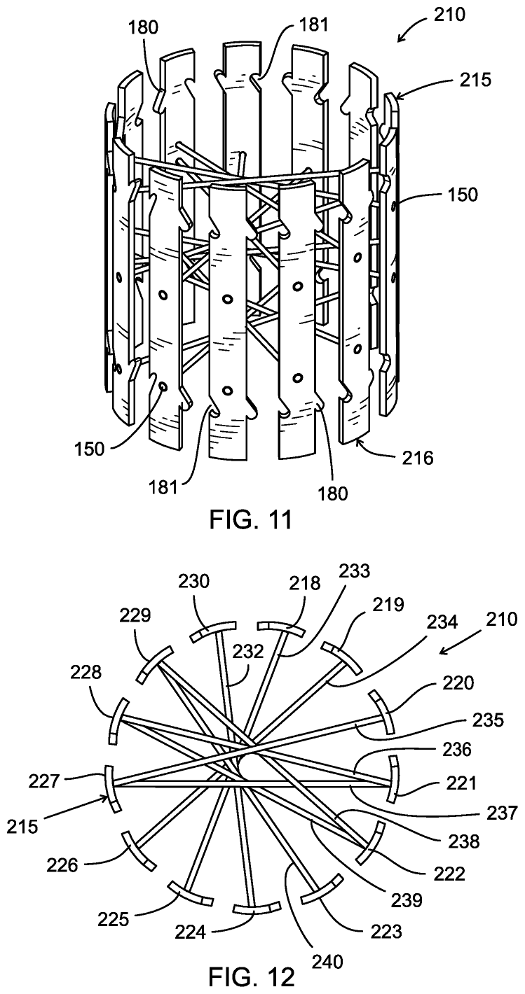

Vascular implant 110 comprises multiple wall panel/wall segments 118-125. For example, as shown in FIGS. 3-5, vascular implant 110 can have eight such wall panels/wall segments 118-125. However, the precise number of wall panels can vary depending on the size of the implant and dimensions desired, both of the implant and of each wall panel. Each wall panel in vascular implant 110 can be connected to at least one other wall panel via one or more filtering members or struts 132. In this embodiment, the wall panels are connected to form respective sets, in this example each set comprises two wall panels connected via one or more filtering members or struts 132. The filtering members 132 can connect two diametrically opposed wall panels, such as wall panel 119 and wall panel 123, as shown in FIG. 3. However, as seen for example in FIGS. 11-13 and 15-17, in various other embodiments, the filtering members are not required to connect to only diametrically opposing wall panels and can connect to more than one wall panel. In FIGS. 3-5, there may be eight such filtering members 132 connecting the various wall panels 118-125. The heights of the filtering members 132 may be staggered on the wall panels 118-125 at various heights to prevent collision of members, forming a mesh-like structure.

One, more than one, in an embodiment all, of the wall panel/wall segments 118-125 may be movable. The wall panel/wall segments 118-125 may be movable, e.g. slidable, with translational movement in the longitudinal direction of the filtering member, corresponding in this embodiment to the radial direction of the implant body 11. The wall panel/wall segments 118-125 can be movable in the radial direction over a range between a more nearby position closest to, but at a distance from, the central axis 17 and a remote position further away from the central axis than the more nearby position. By moving the wall panel/wall segments 118-125, the diameter of the implant body can thus be varied, between a smallest diameter and a largest diameter.

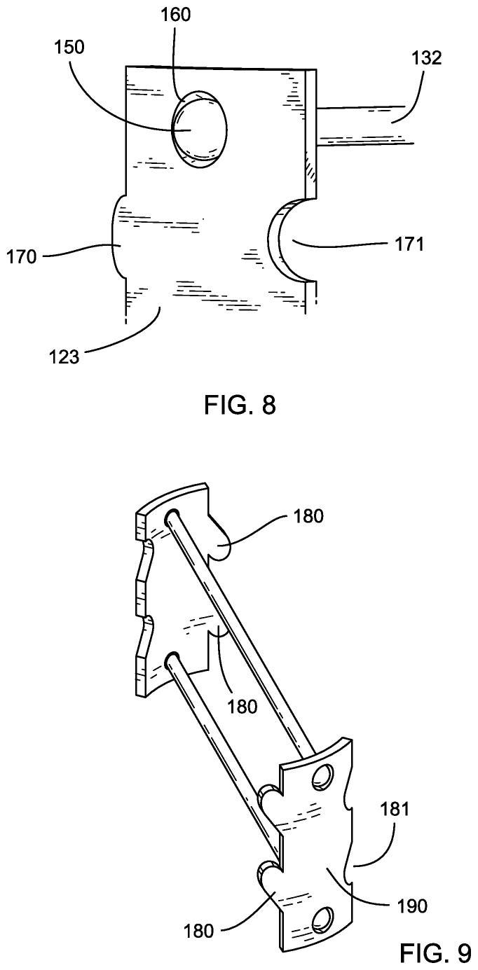

Each filtering member 132 of vascular implant 110 may have a rounded, enlarged end/ball 150 at each end. Each ball 150 prevents each wall panel 118-125 from expanding wider than the length of the filtering member 132. The enlarged end may in other embodiments have another shape than ball, and block the wall panel 118-125 from sliding off the end when the wall panel is moved radially outwards. At the same time, each enlarged end/ball 150 allows for each wall panel 118-125 to move radially inward towards a central axis, as seen by the close-up of panel 123 in FIG. 8 (see how socket 160 permits the wall panel 123 to move inward but not outward).

Some or all of the filtering members 132 may extend, from the open ended bore or passageway 14, through the respective wall panel/wall segment 118-125, beyond the outer surface of the wall panel/wall segment 118-125, with the end of the filtering member 132 projecting radially outwards, at least when vascular implant 110 is partially collapsed and the segments 118-125 are in the more nearby position.

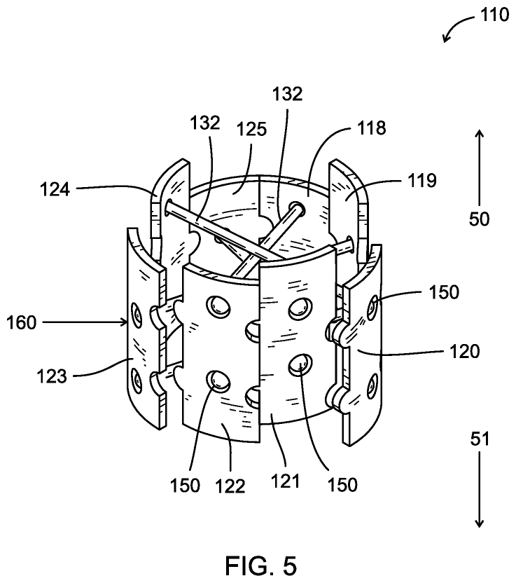

In an embodiment one or more, in this example all, of the wall segments/wall panels 118-125 are provided with a hole/socket 160 through which the filtering member 132 extends. Hole/socket 160 may have a largest diameter smaller than the largest diameter of the enlarged end, such that the end 150 cannot pass through the hole/socket 160. The hole/socket 160 through which the filtering member 132 passes may be conically or frusto-conically shaped, as depicted in FIG. 8, to allow for a change of angle of each filtering member 132 relative to segments 118-125 and accordingly, each wall section/panel 118-125. The wall panel/wall segments 118-125 of vascular implant 110 thus are also movable in an axial direction, as seen in FIG. 5. When travelling to the deployment location, vascular implant 110 is initially in a constrained position. Upon deployment, vascular implant 110 assumes the preferred position shown in FIG. 4. In its constrained position, the wall panels of vascular implant 110 positioned so that diametrically opposed wall panel/wall segments 118,122, 119,123, 120,124 and 121,125 are in axially opposed directions, such that when one wall panel is positioned “upward” (see arrow 50), the opposing wall panel is positioned “downward” (see arrow 51). When in a constrained position, one wall panel of each diametrically opposed wall panels pair is in an “upward” position and the other panel in each pair is in a “downward position.” Upon deployment, the wall panels realign so that they may engage with each other, as seen in FIG. 4. For example, diametrically opposing wall panels can each move towards a central axis, with one panel moving downward and the other upward, until each panel is at a parallel height. Alternatively, one panel can move axially towards the other panel. For example, the panels that are lower, such as panels 118, 125, 123 and 120 can move upward until they reach the height of their diametrically opposing panels 122, 121, 119, 124. Alternatively, the panels that are higher can move downward until they reach the lower height of their diametrically opposing panels. FIG. 5 thus represents vascular implant 110 after it has been deployed, but prior to obtaining its fully expanded configuration shown in FIG. 4. As the wall panels realign so as to be able to engage with one another as shown in FIG. 4, vascular implant 110 moves from a more constrained to a more expanded configuration with respect to its diameter; vertically, the vascular implant 110 moves from a more expanded position to a more constrained position as the wall panels realign and engage with one another.

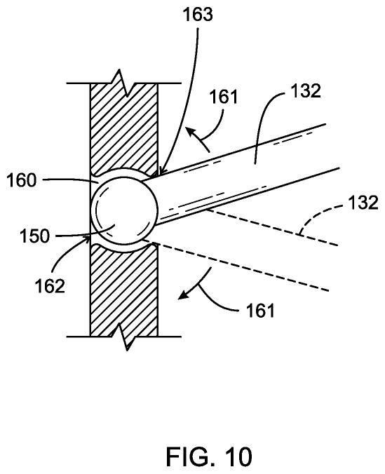

Although the hole/socket 160 has been described as allowing each strut 132 to move in a radial direction, in a more preferred embodiment, the socket 160 is shaped not only to prevent each wall panel from extending outwardly past each ball/rounded portion, but also to prevent each wall panel from extending inwardly past each ball/rounded portion. Such a socket 160 is shown in FIG. 10. That is, the socket 160 can be shaped to encapsulate or capture the ball/rounded portion 150 so as to permit axial, but not radial, movement of each strut 132 (see arrows 161 in FIG. 10). Thus, socket 160 includes openings 162, 163 that each have a diameter that is smaller than the diameter of ball/end portion 150. In such embodiments, where radial but not axial movement of each strut is limited, vascular implant 110 is better able to reassume its preferred position (see FIG. 4) after deployment.

If the vascular implant is on the higher end of the durometer range, for example when the material has a hardness that is within a durometer reading of between about 65 Shore A and 75 Shore D, the vascular implant preferably has ball 150 and socket 160 connections/feature.

Each wall panel can also be connected laterally to adjacent wall panels. For example, as depicted in FIGS. 3-4, each wall panel can interlock with the adjacent wall panels at interlocking portions 170, 171 that can be protrusion 170 and recess 171. In FIG. 3, vascular implant 110 is shown as an expanded view of FIG. 4 in order to detail how the interlocking panels are positioned when engaging with one another. The interlocking portions 170, 171 are in this embodiment on the lateral sides of the respective wall panel, with the protrusion 170 projecting from the lateral side towards the adjacent wall panel. In this embodiment, the wall panels interlock when the vascular implant 110 is in the deployed and interlocked state, with the wall panels at their more expanded position, as seen in FIG. 4. In this embodiment, when the wall panels are not in the most expanded position, the wall panels do not interlock, as seen in FIG. 5.

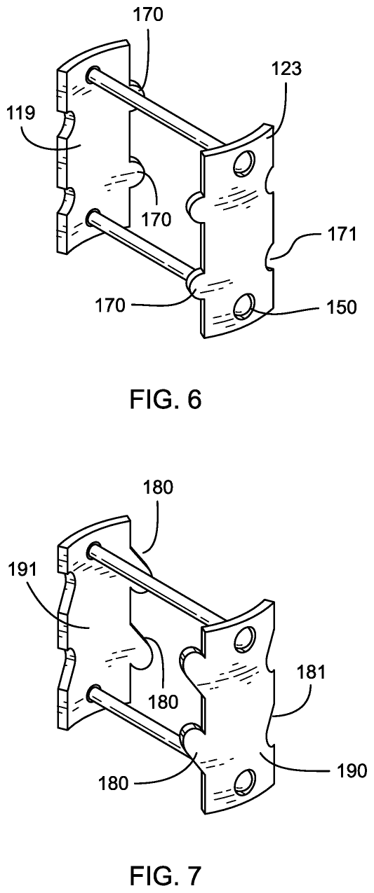

Each wall panel preferably has at least one protrusion 170 on one lateral side and at least one recess 171 for receiving a corresponding protrusion from an adjacent wall panel. For example, as shown in FIGS. 3 and 4, each wall panel may have two wall protrusions 170 which interlock with a corresponding recess 171 of the adjacent wall panel. The shape of the wall protrusion and corresponding recess can vary. For example, as shown in FIGS. 7 and 9, each protrusion 180 and recess 181 can be sloped so as to better prevent separation when compared with directly horizontal and symmetrical protrusion 170 shown in FIG. 6. Preferably, the direction of the slope (either upward or downward) is determined by whether the wall panel is receiving an adjacent wall panel coming from below (in which case the protrusions of the “upward” panel will be sloped downward, as will the recesses of the adjacent “downward” panel) or above (in which case the protrusions of the “downward” panel will be sloped upward, as the corresponding recesses of the “upward” panel will be sloped upward). For example, as seen in FIG. 9, wall panel 190 has downward sloped recesses 181 for receiving protrusions from a wall panel located above it and wall panel protrusions 180 sloped upward for inserting in a wall panel located above it.

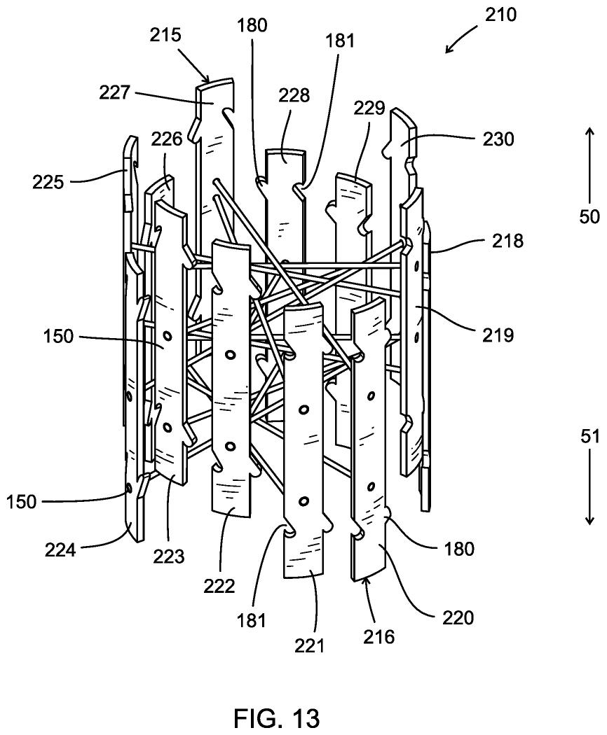

FIGS. 11-14 show a fourth embodiment of the apparatus of the present invention designated generally by the numeral 210. Vascular implant 210 is similar to the vascular implants 10 and 110, except as depicted and noted. Vascular implant 210 has proximal end portion 215 and distal end portion 216. Vascular implant 210 features a plurality of wall panels 218-230, of similar dimensions. However, the wall panels need not be of similar dimensions, as is the case with vascular implant 10 as shown in FIG. 2, where the wall panels have different widths (wall panel 27 is wider than wall panel 26, for example). The interlocking panels of vascular implant 210 each may possess one or more protrusions 180 and recesses 181. As shown in FIG. 11, an exploded view of vascular implant 210, a set of interlocking panels of the vascular implant 210 may have a distal end protrusion of a respective interlocking panel and a corresponding recess on the other interlocking panel, and a proximal end protrusion on a respective interlocking panel and corresponding recess on the other interlocking panel. As noted with respect to FIG. 9, the direction of each protrusion and recess (whether downward or upward) is determined by the direction of the wall panel adjacent each protrusion or recess. For example, as seen in FIG. 12, depicting vascular implant 210 as it transforms from a fully constrained to the fully expanded position depicted in FIG. 13, wall panel 228 is below wall panel 227. Thus, wall protrusions 180 of wall panel 228 are sloped upwardly, as is each corresponding recess of wall panel 227. Wall panel 230, however, is upward of wall panel 229. Thus, each protrusion 180 is sloped downwardly on wall panel 230 as is each recess of wall panel 229. Each protrusion is sloped in the direction of the adjacent wall panel that it is being received by (upward if the adjacent panel is upward, downward if the adjacent panel is downward) and correspondingly, each recess of each wall panel is shaped so as to receive the adjacent protrusion. The distal protrusion and the proximal end protrusion may be on the same interlocking panel, or a panel may have a one of the proximal end protrusion and the distal end protrusion and one of the proximal end recess and the distal end recess. Although each distal end protrusion and corresponding recess and proximal end protrusion and corresponding recess are shown at the same height, they need not be. In this embodiment, protrusions/recesses lie closer to the respective one of the proximal end portion 215 and distal end portion 216 than the position at which the respective filtering members 232 attach to the wall panel. Thereby, in the compressed state a rigid implant body 11 can be obtained.

A respective wall panel 218-230 may be connected to one or more opposing wall panels via filter members/struts 232-240. For example, as shown in FIG. 12, a single wall panel, i.e. 229, can be connected to two opposing panels, 223, 222, via filtering members 238, 240. A wall panel may only be connected to a single opposing panel, such as seen by wall panels 218 and 225, connected by filtering member 233. A wall panel may be part of a set of three of more wall panels connected via successive filtering members 233, such as seen by wall panels 220,221, 222,223, 228,229 for example, which are held together by a successive filtering members 233 connected to different wall panels of the set to connect the wall panels to each other. Wall panel 221 for example is connected to wall panels 227 and 228 via filtering members 236,237 whereas wall panel 227 is in turn connected to wall panel 220 via filtering member 235, and wall panel 228 to wall panel 220 via filtering member 239, etc. Said differently, in the set the wall panels are all, directly or indirectly, linked to each other via the filtering members of the set. The height of each filtering member/strut 232-240 can be staggered so as to prevent collision of the various members 232-240. Each filtering member 232-240 can be connected to each wall panel 218-225 preferably with an enclosed ball and socket connection (e.g. see FIG. 10) wherein each filtering member is able to move axially, but not radially. In alternative embodiments, each filtering member passes through a conically or frusto-conically shaped hole (see FIG. 8) which allows for sliding/pivoting movement of the filtering members 232-240 relative to each wall panel 218-225.

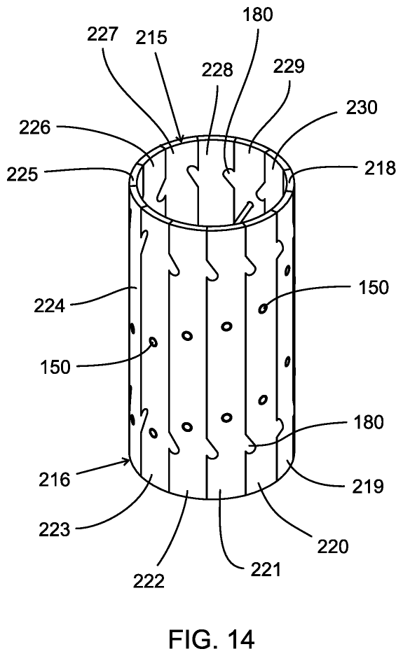

FIG. 13 is a side perspective view of vascular implant 210 depicting axial movement of the wall panels 218-230 during deployment of the vascular implant 210. In the present invention, the vascular implant, prior to deployment, is in a constrained state with the wall panels closer to the central axis than when in a deployed state. In this constrained state, the diameter of the vascular implant is such that it can be transported to the destination site. Preferably, the wall panels move axially, with opposing and connecting wall panels moving in opposite directions (with respect to each other). For example, as seen in FIG. 13, when deployed and moving from a constrained state to a fully expanded state, wall panels 220 and 221 move upwardly towards the direction of proximal end portion 215 until such panels are even with their adjacent panels 219 and 222, and interlock in position. If the length of the filtering members/struts stays constant, as the lower panels move vertically upward towards proximal end portion 215, and as each panel is therefore brought closer to a central axis, the diameter of the vascular implant expands until each wall panel joins with the adjacent wall panels. At this point, the outer face of each wall panel engages with the vessel wall. Thus, for example, when vascular implant 210 is deployed, the wall panels 218-230 can move towards a central plane as the distal end portion 216 of each wall panel moves to occupy the same plane (and similarly the proximal end portion 215 of each panel also moves to occupy a same, but different plane). When the panels line up, they interlock, as shown in FIG. 14. When fully interlocked, as shown in FIG. 14, there are no gaps between adjacent wall panels.

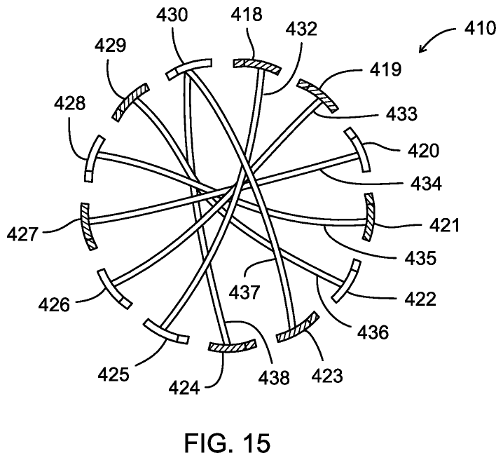

FIG. 15 is a top view of another preferred embodiment of the apparatus of the present invention, designated generally by the numeral 410. Vascular implant 410 is similar to the vascular implant 210, except as depicted and noted. Namely, in vascular implant 410, filtering members/struts 432-438 flex slightly as they pass above and below each other, forming a woven-like filtering structure. As shown in FIG. 15, wall panels/wall segments 418-430 are connected to one or more opposing wall panels via filter members/struts 432-238. In order for the filtering members/struts to flex as shown in vascular implant 410 of FIG. 15, the implant is preferably at the lower end of the durometer range, for example from 10 Shore A to 40 Shore A. In FIG. 15, certain wall panels are marked with partial shading and others without any partial shading to depict which wall panels are shifted downwardly in a vertical direction when vascular implant 410 is in its constrained state and when it moves from its constrained to fully deployed and expanded state. For example, partially shaded wall panels 418, 419, 421, 423, 424, 427 and 429 may move upwards as non-shaded panels 420, 422, 425, 426, 428, and 430 move downwards, or the partially shaded panels may move upwards as the non-shaded panels do not move. Specifically, wall panels connected by filtering members/struts 432-438 may move in opposite directions axially when deploying the vascular implant 410. For example, when deploying vascular implant 410 from a collapsed state, wall panels 424 and 423 may move downward towards a central plane as opposing panel 430 moves upward towards the central plane. In this manner, the diameter of vascular implant increases until the wall panels are at the same height, for example, as seen in FIGS. 10, 13.

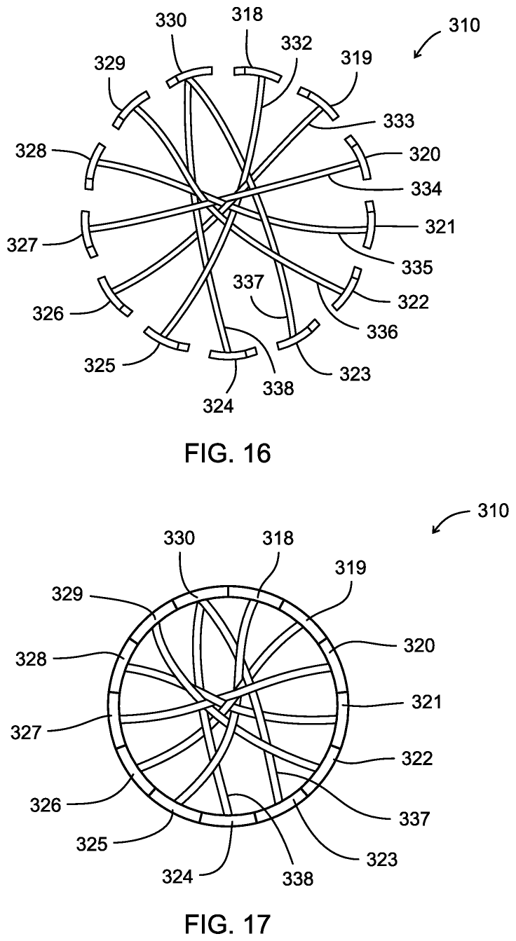

FIGS. 16-17 show another preferred embodiment of the apparatus of the present invention designated generally by the numeral 310. Vascular implant 310 is similar to vascular implant 210 shown in FIGS. 11-14, except as depicted and noted. Namely, in vascular implant 310, filtering members/struts 332-338 flex slightly as they pass above and below each other, forming a woven-like filtering structure. As shown in FIGS. 16-17, wall panels/wall segments 318-330 are connected to one or more opposing wall panels via filter members/struts 332-338. In order for the filtering members/struts to flex as shown in vascular implant 310 of FIGS. 15-16, the implant is preferably at the lower end of the durometer range, for example from 10 Shore A to 40 Shore A. Vascular implant 310 is similar to vascular implant 410, but the filtering members of vascular implant 310 are more interwoven than in vascular implant 410. For instance, whereas in vascular implant 410 filtering member 437 connecting wall panels 423 and 430 passes over the other filtering members, and filtering member 438 connecting wall panels 424 and 430 passes underneath the other filtering members, in vascular implant 310 corresponding filtering member 337 connecting wall panels 323 and 330 crosses some filtering members above and the other filtering members underneath in an alternating manner, and filtering member 338 connecting wall panels 324 and 330 crosses filtering member 333 above and underneath the other filtering members. The degree of flex and degree to which the filtering members are woven can vary and is not limited to what is depicted in FIGS. 15-17. FIGS. 15-17 are mere examples of preferred embodiments. In other embodiments, more or fewer filtering members may be utilized.

Although the filtering members are shown to bend/flex only in those embodiments depicted in FIGS. 15-17, such bending/flexing can exist in a number of other embodiments, included those other depicted embodiments, as long as the hardness is on the lower end of the durometer range, for example from 10 Shore A to 40 Shore A.

Although not pictured, certain wall panels in the various embodiments may possess anchors on the outer walls, similar to those shown in PCT App. No. PCT/US21/73176, filed 30 Dec. 2021, incorporated herein by reference. The anchors are preferably only on those panels which do not move upon deployment (for example, if the lower wall panels of a vascular implant move upwardly after deployment in order to interlock with the upward panels, but the upward panels do not move, the upward panels may have anchors on the outside of the wall panel so as to engage with the vessel wall). In this way, anchors are permitted on approximately half of the wall panels in any given embodiment.

In various embodiments, the hardness of the material utilized for the vascular implant may vary. Preferably, when used in those embodiments without ball and socket features, the material is on the lower end of the durometer range, for example, the material may have a hardness that is within a durometer reading of between 10 Shore A and 40 Shore A. However, when ball and socket connections are utilized, the material may be in the full durometer range, for example, from about 10 Shore A to 75 Shore D. When ball and socket connections are utilized, the material is preferably on the higher end of the durometer range (though need not be), for example when the material has a hardness that is within a durometer reading of between about 65 Shore A and 75 Shore D.

In various embodiments of the present invention, the ball and socket connection can be such that the wall panels can move both axially and radially. However, it is preferable that the socket is constructed in such a manner so that axial movement is permitted, but not radial movement.

The vascular implant can thus be characterized by comprising a tubular implant body having opposed filter ends and a central longitudinal axis, the tubular implant body having a side wall surrounding a central, open ended bore. Multiple filtering elements extend from a position on the side wall through the central, open ended bore to a termination, some of the filtering elements can form an acute angle with the central longitudinal axis.

Without limitation, the implant may further be characterized by one or more of the following statements.
Statement 1. A vascular implant, comprising:
a) a tubular implant body having opposed filter ends and a central longitudinal axis;
b) the tubular implant body having a generally tubular side wall surrounding a central, open ended bore;
c) multiple filtering elements that extend from one position on the side wall to another, spaced apart position on the side wall;
d) wherein some of the filtering elements form an acute angle with the central longitudinal axis; and
e) wherein one or more of the filtering elements extend from a first position on the side wall to a second position on the side wall that is spaced from the first position.
Statement 2. The vascular implant of one or more of the preceding statements wherein the implant body is a 3d printed body.
Statement 3. The vascular implant of one or more of the preceding statements wherein one or more of the filtering elements do not touch one or more others of the filtering elements.
Statement 4. The vascular implant of one or more of the preceding statements wherein the side wall comprises an upper end and a lower end and wherein one or more of the filtering elements attaches to the side wall at a position that is close to the upper end and to the side wall at a position that is closer to the lower end.
Statement 5. The vascular implant of one or more of the preceding statements wherein multiple of the filtering elements are attached to the side wall at positions on the side wall less than 180 degrees apart.
Statement 6. The vascular implant of one or more of the preceding statements wherein the side wall is a segmented side wall comprised of multiple circumferentially spaced apart wall sections.
Statement 7. The vascular implant of statement 6 wherein each the wall section has a generally rectangular shape.
Statement 8. The vascular implant of one or more of the preceding statements wherein the opposed filter ends include a proximal end and a distal end.
Statement 9. The vascular implant of one or more of the preceding statements wherein the body is of a material that is too soft to be machined.
Statement 10. The vascular implant of one or more of the preceding statements wherein the implant body has a durometer reading of between 35 Shore A and 75 Shore D. Statement 11. The vascular implant of one or more of the preceding statements wherein the implant body is of a resorbable material that resorbs when exposed to the human body vascular system.
Statement 12. The vascular implant of statement 11 wherein the implant body is of bioresorbable polymeric material.
Statement 13. The vascular implant of one or more of the preceding statements wherein one or more of the filtering elements connects to the side wall and another the filtering element.
Statement 14. A vascular implant, comprising:
a) a tubular implant body having opposed filter ends and a central longitudinal axis;
b) the implant body having a segmented side wall comprised of spaced apart wall panels that surround an open-ended bore that extends along the central longitudinal axis;
c) multiple filtering elements that extend to a core of the implant body, some of the filtering elements terminating at the core where they connect to another the filtering element;
d) wherein some of the filtering elements form an acute angle with the central longitudinal axis; and
e) wherein one or more the filtering elements connects to the side wall with a ball and socket connection.
Statement 15. The vascular implant of statement 14 wherein the implant body is a 3d printed body.
Statement 16. The vascular implant of statement 14 or 15 wherein the side wall comprises an upper end and a lower end and wherein one or more of the filtering elements attaches to the side wall at a position that is close to the upper end and to the side wall at a position that is closer to the lower end.
Statement 17. The vascular implant of statement 14, 15 or 16 wherein the body is of a material that is too soft to be machined.
Statement 18. The vascular implant of statement 14, 15, 16, 17 wherein the implant body is of a resorbable material that resorbs when exposed to the human body vascular system.
Statement 19. The vascular implant of statement 18 wherein the implant body is of bioresorbable polymeric material.
Statement 20. A vascular implant, comprising:
a) a tubular implant body having opposed filter ends, a central longitudinal axis;
b) the implant body having a segmented side wall comprised of spaced apart wall panels that surround an open ended bore that extends along the central longitudinal axis;
c) the implant body has a core portion spaced between the opposed filter ends and spaced inwardly of the segmented side wall;
d) multiple filtering elements that extend from the side wall to the core of the filter body, some of the filtering elements terminating at the core where they connect to another the filtering element; and
e) wherein one or more the filtering elements extend from one the wall segment to another of the wall segments.

The following is a list of parts and materials suitable for use in the present invention.

PARTS LIST

| Part Number | Description |
|---|---|
| 10 | vascular implant |
| 11 | implant body |
| 12 | vascular tissue |
| 13 | side wall/tubular segmented wall |
| 14 | open ended bore/passageway |
| 15 | first end portion/proximal end portion |
| 16 | second end portion/distal end portion |
| 17 | central longitudinal axis |
| 18 | wall panel/wall segment/wall section |
| 19 | wall panel/wall segment/wall section |
| 20 | wall panel/wall segment/wall section |
| 21 | wall panel/wall segment/wall section |
| 22 | wall panel/wall segment/wall section |
| 23 | wall panel/wall segment/wall section |
| 24 | wall panel/wall segment/wall section |
| 25 | wall panel/wall segment/wall section |
| 26 | wall panel/wall segment/wall section |
| 27 | wall panel/wall segment/wall section |
| 28 | wall panel/wall segment/wall section |
| 29 | wall panel/wall segment/wall section |
| 30 | wall panel/wall segment/wall section |
| 31 | central region/core |
| 32 | filtering member/strut |
| 33 | filtering member/strut |
| 34 | filtering member/strut |
| 35 | filtering member/strut |
| 36 | filtering member/strut |
| 37 | filtering member/strut |
| 38 | filtering member/strut |
| 39 | filtering member/strut |
| 40 | filtering member/strut |
| 41 | filtering member/strut |
| 42 | filtering member/strut |
| 43 | filtering member/strut |
| 44 | filtering member/strut |
| 45 | filtering member/strut |
| 46 | concave inner surface |
| 47 | convex outer surface |
| 48 | gap |
| 50 | arrow |
| 51 | arrow |
| 110 | vascular implant |
| 118 | wall panel/wall segment/wall section |
| 119 | wall panel/wall segment/wall section |
| 120 | wall panel/wall segment/wall section |
| 121 | wall panel/wall segment/wall section |
| 122 | wall panel/wall segment/wall section |
| 123 | wall panel/wall segment/wall section |
| 124 | wall panel/wall segment/wall section |
| 125 | wall panel/wall segment/wall section |
| 132 | filtering member/strut |
| 150 | filtering member end/ball |
| 160 | hole/socket |
| 161 | arrow |
| 162 | opening |
| 163 | opening |
| 170 | protrusion/interlocking portion |
| 171 | recess/interlocking portion |
| 180 | protrusion/interlocking portion |
| 181 | recess/interlocking portion |
| 190 | wall panel/wall segment |
| 191 | wall panel/wall segment |
| 210 | vascular implant |
| 215 | proximal end portion |
| 216 | distal end portion |
| 218 | wall panel/wall segment/wall section |
| 219 | wall panel/wall segment/wall section |
| 220 | wall panel/wall segment/wall section |
| 221 | wall panel/wall segment/wall section |
| 222 | wall panel/wall segment/wall section |
| 223 | wall panel/wall segment/wall section |
| 224 | wall panel/wall segment/wall section |
| 225 | wall panel/wall segment/wall section |
| 226 | wall panel/wall segment/wall section |
| 227 | wall panel/wall segment/wall section |
| 228 | wall panel/wall segment/wall section |
| 229 | wall panel/wall segment/wall section |
| 230 | wall panel/wall segment/wall section |
| 232 | filtering member/strut |
| 233 | filtering member/strut |
| 234 | filtering member/strut |
| 235 | filtering member/strut |
| 236 | filtering member/strut |
| 237 | filtering member/strut |
| 238 | filtering member/strut |
| 239 | filtering member/strut |
| 240 | filtering member/strut |
| 310 | vascular implant |
| 318 | wall panel/wall segment/wall section |
| 319 | wall panel/wall segment/wall section |
| 320 | wall panel/wall segment/wall section |
| 321 | wall panel/wall segment/wall section |
| 322 | wall panel/wall segment/wall section |
| 323 | wall panel/wall segment/wall section |
| 324 | wall panel/wall segment/wall section |
| 325 | wall panel/wall segment/wall section |
| 326 | wall panel/wall segment/wall section |
| 327 | wall panel/wall segment/wall section |
| 328 | wall panel/wall segment/wall section |
| 329 | wall panel/wall segment/wall section |
| 330 | wall panel/wall segment/wall section |
| 332 | filtering member/strut |
| 333 | filtering member/strut |
| 334 | filtering member/strut |
| 335 | filtering member/strut |
| 336 | filtering member/strut |
| 337 | filtering member/strut |
| 338 | filtering member/strut |
| 410 | vascular implant |
| 418 | wall panel/wall segment/wall section |
| 419 | wall panel/wall segment/wall section |
| 420 | wall panel/wall segment/wall section |
| 421 | wall panel/wall segment/wall section |
| 422 | wall panel/wall segment/wall section |
| 423 | wall panel/wall segment/wall section |
| 424 | wall panel/wall segment/wall section |
| 425 | wall panel/wall segment/wall section |
| 426 | wall panel/wall segment/wall section |
| 427 | wall panel/wall segment/wall section |
| 428 | wall panel/wall segment/wall section |
| 429 | wall panel/wall segment/wall section |
| 430 | wall panel/wall segment/wall section |
| 432 | filtering member/strut |
| 433 | filtering member/strut |
| 434 | filtering member/strut |
| 435 | filtering member/strut |
| 436 | filtering member/strut |
| 437 | filtering member/strut |
| 438 | filtering member/strut |

All measurements disclosed herein are at standard temperature and pressure, at sea level on Earth, unless indicated otherwise. All materials used or intended to be used in a human being are biocompatible, unless indicated otherwise.

The foregoing embodiments are presented by way of example only; the scope of the present invention is to be limited only by the following claims.

1. A vascular implant, comprising:
a) a tubular implant body having opposed filter ends and a central longitudinal axis:
b) said tubular implant body having a generally tubular side wall that is comprised of spaced apart panels, each said panel having panel ends and panel edges, said tubular side wall surrounding a central, open ended bore:
c) multiple filtering elements that extend from one position on said side wall panel to another, spaced apart position on a said side wall panel:
d) wherein some of said filtering elements form an acute angle with said central longitudinal axis; and
e) wherein one or more of said filtering elements extend from a first position on said side wall panel to a second position on another said side wall panel that is spaced from said first position.

2. The vascular implant of claim 1 wherein said implant body is a 3d printed body.

3. The vascular implant of claim 1 wherein one or more of said filtering elements do not touch one or more others of said filtering elements.

4. The vascular implant of claim 1 wherein said side wall comprises an upper end and a lower end and wherein one or more of said filtering elements attaches to said side wall at a position that is close to said upper end and to said side wall at a position that is closer to said lower end.

5. The vascular implant of claim 1 wherein multiple of said filtering elements are attached to said side wall at positions on said side wall less than 180 degrees apart.

6. The vascular implant of claim 1 wherein said side wall is a segmented side wall comprised of multiple circumferentially spaced apart curved wall panels.

7. The vascular implant of claim 6 wherein each said wall section panel has a generally rectangular shape.

8. The vascular implant of claim 1 wherein said opposed filter ends include a proximal end and a distal end.

9. The vascular implant of claim 1 wherein the body is of a material that is too soft to be machined.

10. The vascular implant of claim 1 wherein the implant body has a durometer reading of between 35 Shore A and 75 Shore D.

11. The vascular implant of claim 1 wherein the implant body is of a resorbable material that resorbs when exposed to the human body vascular system.

12. The vascular implant of claim 11 wherein the implant body is of bioresorbable polymeric material.

13. The vascular implant of claim 1 wherein one or more of said filtering elements connects to said side wall and another said filtering element.

14. A vascular implant, comprising:
a) a tubular implant body having opposed filter ends and a central longitudinal axis:
b) said implant body having a segmented side wall comprised of spaced apart wall panels that surround an open ended bore that extends along said central longitudinal axis:
c) multiple filtering elements that extend to a core of said implant body, some of said filtering elements terminating at said core where they connect to another said filtering element:
d) wherein some of said filtering elements form an acute angle with said central longitudinal axis; and
e) wherein one or more said filtering elements connects to said side wall panel with a ball and socket connection.

15. The vascular implant of claim 14 wherein said implant body is a 3d printed body.

16. The vascular implant of claim 14 wherein said side wall comprises an upper end and a lower end and wherein one or more of said filtering elements attaches to said side wall at a position that is close to said upper end and to said side wall at a position that is closer to said lower end.

17. The vascular implant of claim 14 wherein the body is of a material that is too soft to be machined.

18. The vascular implant of claim 14 wherein the implant body is of a resorbable material that resorbs when exposed to the human body vascular system.

19. The vascular implant of claim 18 wherein the implant body is of bioresorbable polymeric material.

20. A vascular implant, comprising:
a) a tubular implant body having opposed filter ends, a central longitudinal axis;
b) said implant body having a segmented side wall comprised of multiple spaced apart wall panels that surround an open ended bore that extends along said central longitudinal axis:
c) said implant body has a core portion spaced between said opposed filter ends and spaced inwardly of said segmented side wall:
d) multiple filtering elements that extend from said side wall to said core of said filter body, some of said filtering elements terminating at said core where they connect to another said filtering element; and
e) wherein one or more said filtering elements extend from one said wall panel to another of said side wall panels.

21-45. (canceled)
